(19) United States
(12) Patent Application Publication
(10) Pub. No.: US 2025/0241572 A1
(43) Pub. Date: Jul. 31, 2025

(54) LIQUID AND/OR PH SENSING DEVICES AND SYSTEMS, AND RELATED METHODS OF USE

(71) Applicant: University of Louisville Research Foundation, Inc., Louisville, KY (US)

(72) Inventors: Cindy HARNETT, Louisville, KY (US); Ji-Tzuoh LIN, Louisville, KY (US)

(73) Assignee: UNIVERSITY OF LOUISVILLE RESEARCH FOUNDATION, INC., Louisville, KY (US)

(21) Appl. No.: 19/036,146

(22) Filed: Jan. 24, 2025

Related U.S. Application Data
(60) Provisional application No. 63/624,980, filed on Jan. 25, 2024.

(51) Int. Cl. A61B 5/20; A61B 5/00

(52) U.S. Cl. CPC A61B 5/202; A61B 5/0059; A61B 5/6891

(57) ABSTRACT

Certain aspects of this invention relate to liquid sensing devices and systems that are reusable and washable multiple times without loss or significant loss of function. Some devices employ optical waveguides and liquid-and/or pH-sensitive materials which have changing light transmission properties when exposed to liquids and can vary with pH. Some changes in light transmission properties can be used to determine the presence or absence of liquid and, in some devices, the pH or pH range of the liquid present. Additional embodiments of the invention are also disclosed herein.

### CROSS-REFERENCE TO RELATED APPLICATIONS

This application claims the benefit of U.S. Provisional Application No. 63/624,980, filed Jan. 25, 2024 entitled “TEXTILE-INTEGRATED FIBER OPTIC MOISTURE SENSOR” which is herein incorporated by reference in its entirety.

### STATEMENT OF GOVERNMENT INTEREST

This invention was made with government support (a) under ED21HDQ3070047 awarded by the U.S. Economic Development Administration (PREPARe program), (b) under AG078020 awarded by the National Institutes of Health (SBIR program), and (c) under contract no. 1849213 awarded by the National Science Foundation. The government has certain rights in the invention.

### BACKGROUND

Bedwetting and incontinence can be problems, for example, among the young, infirm, and old. Various solutions and products attempting to assist persons and caretakers affected by these issues have been developed. However, there are several problems among such solutions and products. For example, one problem is the reliance on components which are not susceptible to reuse and washability. Another problem is that they can provide a hazard (e.g., a shock or electrocution hazard) to the person and/or metal-based e-textile systems (e.g., metal wires in textiles) can corrode over time with exposure to water, which can lead to problems such as loss of conductivity and increase in local acidity which can degrade the textile. Certain objectives of the invention include avoidance of the typical problems with metal-based e-textile systems which can include, for example, lower temperature sensitivity and immunity to electromagnetic interference. Further, such solutions and products (e.g., metal-based e-textile systems) do not incorporate a means to measure or detect pH (e.g., of a person's bodily fluid), which, in certain instances, can be helpful in ascertaining and/or monitoring a person's condition. In certain embodiments, the invention relates to devices and methods capable of sensing liquids and/or the pH of the liquid, where the liquid and/or the pH sensors can be washed and reused.

Additional embodiments of the invention are also disclosed herein.

### SUMMARY

According to some embodiments, one objective of the invention is a device which is capable of sensing liquids and/or the pH of the liquid, where some or all of the device is capable of being washed and reused. Embodiments of devices include liquid sensors (e.g., using fiber optics with hydrochromic ink), where the sensors are machine washable and dryable and which when in a state of use are combined with one or more light sources and electronics that reversibly attach (e.g., magnetically) after washing. The hydrochromic ink may be or include a material such as but not limited to bromothymol blue which changes light transmission properties based not only on the presence vs absence of liquid, but also based on the pH of a liquid when liquid is present. A washable ink may be incorporated (e.g., locked) into a porous structure to extend the life of the device while still giving it access to fluids. The color change (e.g., changes in light transmission intensities at one or more wavelengths) of exemplary hydrochromic inks used in exemplary sensors are reversible (e.g., after one or more washings and dryings the sensors). Exemplary devices may be subject to cyclic washing and reuse and, in some embodiments, cyclic mechanical drying as well.

Some exemplary embodiments provide liquid sensing devices and systems which are reusable and washable multiple times without loss or significant loss of function. Some embodiments entail frequently washable pads for bedding. Some exemplary embodiments comprise liquid sensors configured for reuse and washability. Some exemplary embodiments provide pH detection that extends the liquid sensing capability to pH evaluation embedded in the same sensor. The pH indication may be used as a reference either for further urinalysis needs or monitoring patients' dieting or medication changes.

Exemplary embodiments may comprise optical fiber and coating combinations configured to maintain sensitivity and accuracy through multiple washings, and in applications where users may be expected to press on the sensor inadvertently, such as bedding applications. For the latter concern, exemplary embodiments may be configured to decouple signals caused by liquid from those caused by pressure. A differential approach, for example where the signals are compared between a pair of water-reactive coated fibers and a pair of uncoated fibers experiencing the same pressure signal, may be employed in some embodiments overcome this technical hurdle.

Exemplary embodiments may detect liquid alone, pH, or a combination of liquid and pH. For instance, an exemplary embodiment may be a liquid sensor or smart pad incorporating an embedded liquid sensor which is configured for urine pH detection.

Exemplary products such as but not limited to bedding may include pH detection of urine and/or other fluids. With urine, a pH value greater than 9 is generally correlated with urinary tract infections, where rapid detection and treatment is typically needed to prevent kidney involvement. pH is a key indicator of other urinary tract related conditions such as kidney stones and drug use. Persistently alkaline urine is an indication for complete urinalysis and urine culture. In conjunction with other specific urine and plasma measurements, urine pH is often invaluable in diagnosing systemic acid-base disorders. In the current standard of care, bed wetting monitoring is a basic preventive measure to prevent bed sores. However, existing wetting monitor solutions ignore pH entirely. By contrast, some exemplary embodiments are specifically configured to detect the pH of fluid to which they are exposed. Adding pH monitoring to these systems is likely to benefit, e.g., the same group of patients who benefit from wetting monitoring, providing a rapid warning sign indicating to caretakers the need for further urinalysis. pH status may be ascertained through comparison of the intensity from discrete light sources and selective detectors, for example. An exemplary sensor may distinguish dry, wet, acidic, and basic (e.g., pH>9) conditions.

Exemplary optical sensor embodiments offer multiple advantages over electronic sensors. An optical sensor element comprising or consisting exclusively of polymer differs from existing textile-based liquid sensors that use electronic resistance. Electronic sensors have greater temperature drift than optical sensors because conductivity changes with temperature in most materials. Resistive sensors contain conductive films that interact directly with liquids, but the films' electronic properties change if the materials oxidize (tarnish), in contrast to soft optical sensors which do not have this drawback. Laundry cycles create conditions for metal oxidation with water, surfactants, and heat, so soft electronic liquid sensors are generally one-time use disposables such as diapers, or wipe-clean plastic mats. In contrast, an optical sensor may be embodied as a textile-embedded, washable sensor that may be used, washed, and re-used multiple times.

Exemplary applications for present embodiments include but are not limited to: detecting liquid in hospital beds, in agricultural irrigation monitoring applications where the presence of metal could lead to ingestion hazards, in furniture such as heated car seats where additional metal wiring poses a short-circuit hazard, in magnetic resonance imaging systems where metals interfere with imaging, and in environments with strong electromagnetic interference that disrupts electronic sensors.

Some embodiments of the invention include a washable and re-usable sensing device, comprising a light emitting part; a light collecting part; a gap separating a portion of or all of the light emitting part and a portion of or all of the light collecting part, from one another; a liquid-sensitive material in the gap, wherein one or more light transmission properties of the liquid-sensitive material change based on exposure to a liquid and/or pH of a present liquid; wherein the light collecting part is configured to collect light emitted from the light emitting part which has passed through the liquid-sensitive material and transmit the collected light to at least one terminal configured to connect the light collecting part to one or more light detectors.

In certain embodiments, the washable and re-usable sensing device further comprises the one or more light detectors, wherein the one or more light detectors are configured to detect one or more of light transmissions at one or more wavelengths. In other embodiments, the washable and re-usable sensing device further comprises at least one electronic circuit configured to determine a presence or absence of liquid and/or a pH of a present liquid based on a comparison of detected light transmissions at one or more wavelengths with an initial light transmission at one or more wavelengths characterizing the light emitted from the light emitting part. In still other embodiments, the washable and re-usable sensing device further comprises at least one output device configured to show different visual appearances when the presence of liquid F2ais determined versus when an absence of liquid is determined, and/or when the determined pH exceeds a predetermined threshold versus when the determined pH does not exceed a predetermined threshold. In yet other embodiments, (1) the light emitting part and light collecting part are optical fibers, and (2) (a) the two optical fibers are arranged so that a portion of or all of one or both optical fibers are in parallel in a region of the gap and/or (b) the two optical fibers are substantially coaxial with one another in a region of the gap. In certain embodiments, the liquid-sensitive material comprises a washable hydrochromic ink. In some embodiments, the liquid-sensitive material comprises one or more of a mesh, at least one hydrophilic porous material, or at least one open-cell foam. In other embodiments, the washable and re-usable sensing device further comprises one or more textiles and/or nonwovens in which the light emitting part and light collecting part are embedded. In still other embodiments, the one or more light transmission properties comprise one or more of opacity, transparency, light transmission(s) at one or more wavelengths, the product of light transmissions from more than one wavelength, travel time, or color.

Some embodiments of the invention include a method of sensing a presence of liquid and/or a pH or pH range of a present liquid, comprising: emitting light from a light emitting part toward a light collecting part, wherein a gap separates a portion of or all of the light emitting part and a portion of or all of light collecting part from one another, wherein a liquid-sensitive material is positioned in the gap, wherein one or more light transmission properties of the liquid-sensitive material change based on exposure to the liquid and/or the pH of the liquid; detecting one or more of light transmissions at one or more wavelengths of light collected by the light collecting part; determining a presence or absence of liquid and/or a pH or a pH range of a present liquid based on a comparison of detected light transmissions at one or more wavelengths with an initial light transmission at one or more wavelengths characterizing the light emitted from the light emitting part; and showing different visual appearances at one or more output devices (a) when the presence of liquid is determined, as compared to when an absence of liquid is determined, and/or (b) when (1) the determined pH is in a predetermined pH range and/or (2) the determined pH exceeds a predetermined threshold, as compared to when the determined pH does not exceed a predetermined threshold and/or (3) the determined pH is below a predetermined threshold, as compared to when the determined pH is not below a predetermined threshold.

Other embodiments of the invention include a washable and re-usable liquid sensing device, comprising at least one first optical waveguide configured to transmit light from at least one light source and emit some of the transmitted light through a side or end of the at least one first optical waveguide; at least one second optical waveguide; a liquid-sensitive material positioned between the at least one first optical waveguide and the at least one second optical waveguide such that light emitted from at least a portion of the at least one first optical waveguide must pass through the liquid-sensitive material to reach at least a portion of the at least one second optical waveguide, wherein one or more light transmission properties of the liquid-sensitive material change based on the exposure of the liquid-sensitive material. In certain embodiments, the at least one second optical waveguide is configured to collect incident light emitted from the at least one first optical waveguide which has passed through the liquid-sensitive material and transmit the collected light to at least one light detector terminal.

In some embodiments, the washable and re-usable liquid sensing device further comprises a terminal configured for connection to a light source for admitting light into the at least one first optical waveguide and to a detector for receiving light exiting an end of the at least one second optical waveguide. In certain embodiments, the washable and re-usable liquid sensing device further comprises one or more textiles and/or nonwovens in which the at least one first optical waveguide and the at least one second optical waveguide are embedded. In still other embodiments, the washable and re-usable liquid sensing device is configured as a bedding and/or a pad. In some embodiments, the liquid-sensitive material is configured as at least one coating of a hydrochromic ink on one or more of the at least one first optical waveguide and the at least one second optical waveguide. In yet other embodiments, the liquid-sensitive material is configured as a film layer. In certain embodiments, the at least one first optical waveguide and/or the at least one second optical waveguide is optical fiber comprising PMMA or urethane. In still other embodiments, the liquid-sensitive material comprises one or more of a mesh, at least one hydrophilic porous material, or at least one open-cell foam containing a hydrochromic ink. In some embodiments, the liquid-sensitive material comprises one or more of tungsten disulfide, reduced graphene oxide, or zinc oxide nanoparticles. In other embodiments, the one or more light transmission properties comprise one or more of opacity, transparency, light transmission(s) at one or more wavelengths, the product of light transmissions from more than one wavelength, travel time, or color.

Additional embodiments of the invention are also disclosed herein.

### BRIEF DESCRIPTION OF THE DRAWINGS

The following drawings form part of the present specification and are included to further demonstrate certain aspects of the present invention. The invention may be better understood by reference to one or more of these drawings in combination with the description of specific embodiments presented herein.

FIG. 1A is a block diagram of an exemplary sensing device and system.

FIG. 1B is a block diagram of another exemplary sensing device and system.

FIG. 2 is an exemplary liquid sensor including a liquid-reactive layer and optical fibers. The inter-fiber water reactive structure is reusable, washable, and unobtrusive textile-embedded liquid sensing technology. The water-reactive layer turns a soft polymer fiber optic pair into a liquid sensor compatible with textile fabrication methods.

FIG. 3A is an exemplary multizone detection ribbon sensor.

FIG. 3B shows exemplary multi core couplers/terminals.

FIG. 4A is an exemplary sensor device.

FIG. 4B is a button housing which slips on and aligns a fiber across a gap.

FIG. 5 shows sample reactivity data from an exemplary device.

FIG. 6 is transmission spectroscopy for another exemplary device with a white light emitting diode (LED) light source. The device indicates the acidity of the fluid to which the sensor is exposed.

FIG. 7 is detection results for an exemplary device with a tricolor LED light source emitting primary colors in sequence. The results are data from when the sensor is exposed to water, acidic (CH₃COOH) and basic (NaOH) fluids.

FIG. 8 is an example of a dry sensor button which holds two ends of optical fibers.

FIG. 9 is an example of a wet sensor button which holds two ends of optical fibers.

FIG. 10 is a plot of the transmission (also referred to as transmission intensity) as a function of time when exposed to water using the sensor button of FIGS. 8 and 9.

### DETAILED DESCRIPTION

While embodiments encompassing the general inventive concepts may take diverse forms, various embodiments will be described herein, with the understanding that the present disclosure is to be considered merely exemplary, and the general inventive concepts are not intended to be limited to the disclosed embodiments.

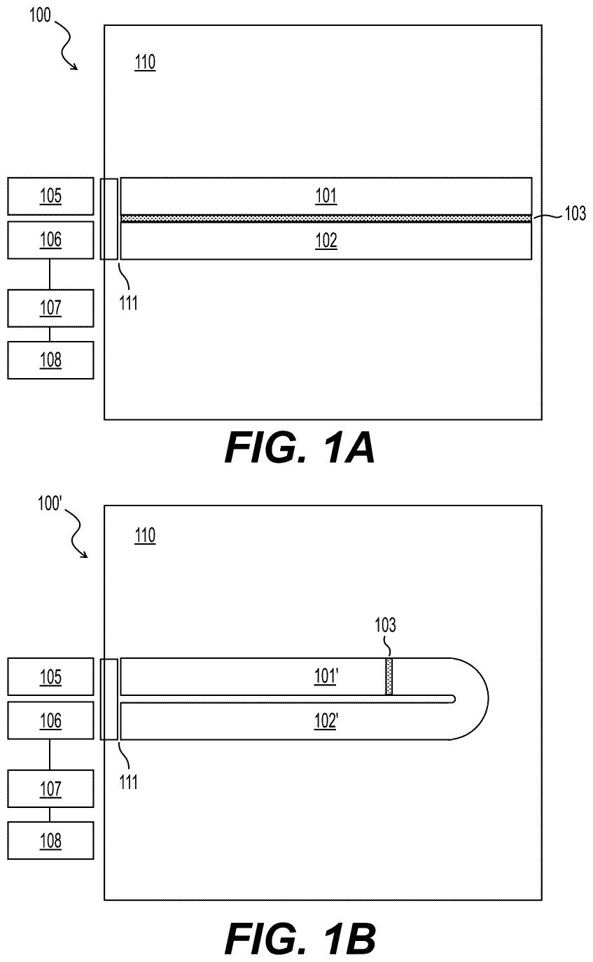

FIG. 1A is an exemplary sensing device 100 depicted in block diagram format. The sensing device 100 comprises a light emitting part 101, a light collecting part 102, a gap separating the parts 101 and 102 from one another, and a sensing material 103 positioned in the gap. Parts 101 and 102 are both light conducting. For example, both parts 101 and 102 may be optical waveguides. As used herein, an optical waveguide is a physical structure that guides electromagnetic waves. The optical waveguide can, in some embodiments, include a coating or cladding on a portion of the optical waveguide or all of the optical waveguide. Any suitable optical waveguides can be used in the devices described here, where such suitability can be determined any number of factors, including but not limited to one or more of: wavelength(s) of light to be used, length of fiber needed, transmissibility, flexibility, configuration of light detecting part (e.g., 101) with light collecting part (e.g., 102) to detect liquid, sensing material (103) (e.g., liquid sensing material, pH sensing material, or both), ability to embed in a fabric (e.g., woven, non-woven, or textile), ability to withstand washing/drying, human feel (e.g., softness) of the fiber optical waveguides in a fabric, ability to be secured to the fabric, and the like. Example optical waveguides include, but are not limited to, optical fibers, glass fibers, quartz optical fibers, polymeric optical fibers (e.g., polymethyl methacrylate (PMMA) fibers or urethane fibers), silicone optical fibers, or hydrogel optical fibers. The length of the light emitting part 101 (e.g., a first optical waveguide) and the length of the light emitting part 102 (e.g., second optical waveguide) can be any suitable lengths, where such suitability can be different for light emitting part 101 and light emitting part 102. Such suitability can depend on any number of factors including but not limited to, light to be used, transmissibility, flexibility, configuration of light detecting part (e.g., 101) with light collecting part (e.g., 102) to detect liquid, sensing material (103) (e.g., liquid sensing material, pH sensing material, or both), ability to embed in a fabric (e.g., woven, non-woven, or textile), ability to withstand washing/drying, human feel (e.g., softness) of the fiber optical waveguides in a fabric, ability to be secured to the fabric, and the like. In certain embodiments, the length of the light emitting part 101 (e.g., a first optical waveguide) and the length of the light emitting part 102 (e.g., second optical waveguide) can be any suitable length, including but not limited to 0.005 meters, 0.01 meters, 0.02 meters, 0.05 meters, 0.1 meters, 0.2 meters, 0.5 meters, 0.7 meters, 1 meter, 10 meters, 20 meters, 30 meters, 40 meters, 50 meters, 60 meters, 70 meters, 80 meters, 90 meters, 100 meters, 125 meters, 150 meters, 175 meters, or 200 meters. In certain embodiments, the diameter of the light emitting part 101 (e.g., a first optical waveguide such as an optical fiber) and the length of the light emitting part 102 (e.g., second optical waveguide such as an optical fiber) can be any suitable diameter, including but not limited to 0.005 mm, 0.01 mm, 0.02 mm, 0.03 mm, 0.04 mm, 0.05 mm, 0.06 mm, 0.07 mm, 0.08 mm, 0.09 mm, 0.1 mm, 0.2 mm, 0.3 mm, 0.4 mm, 0.5 mm, 0.6 mm, 0.7 mm, 0.8 mm, 0.9 mm, 1 mm, 1.1 mm, 1.2 mm, 1.3 mm, 1.4 mm, 1.5 mm, 1.6 mm, 1.7 mm, 1.8 mm, 1.9 mm, 2 mm, 3 mm, 4 mm, 5 mm, 7 mm, or 10 mm.

Light with first characteristics is emitted by the light emitting part 101. The light collecting part 102 is configured to collect light emitted from part 101 which has passed through the material 103, describable as having second characteristics some of which may be different from the first characteristics, and transmit the collected light to at least one terminal/coupler 111 configured to connect the light collecting part to one or more light detectors 106. Light from a light emitting part may or may not be produced by the light emitting part. According to the exemplary device 100 of FIG. 1A, the light emitting part 101 is an optical waveguide, such as an optical fiber, which is configured to receive light from one or more light sources 105. Light with the first characteristics travels through the light emitting part 101, with at least some of the light exiting the light emitting part 101, e.g., from a side or an end of the waveguide, in a direction of the light receiving part 102.

The gap separating parts 101 and 102 from one another can be any suitable size, for example, so that the liquid can enter the gap and can suitably contact and/or interact with the sensing material 103. For example, the gap can be 50 microns, 100 microns, 200 microns, 300 microns, 400 microns, 500 microns, 600 microns, 700 microns, 800 microns, 900 microns, 1 mm, 1.1 mm, 1.2 mm, 1.3 mm, 1.4 mm, 1.5 mm, 2 mm, or 3 mm.

The sensing material 103 can be a liquid sensing material, a pH sensing material or both. The liquid sensed by the liquid sensing material can be any liquid including, but not limited to water, aqueous solutions, intravenous fluids (e.g., from leaky tubing/connections or accidental disconnection), bodily fluids, urine, sweat, saliva, tears, mucus, diarrhea-related fluids, blood, pus, semen, bile, bodily discharge, alcohols, ethanol, propanol, butanol, solvents, acetone, phenol, cleaning fluids, disinfectants, benzene, toluene, acidic solutions (e.g., with weak acids or strong acids), basic solutions (e.g., with weak bases or strong bases), acetic acid, peracetic acid, bleach, chlorine dioxide, hydrogen peroxide, or a combination thereof. The amount of liquid can be any suitable amount of liquid such that the liquid sensing material can detect the liquid, and the amount of liquid can include increases in humidity and the presence of moisture (i.e., very small amounts of liquid). With a liquid sensing material, the material changes properties (e.g., light transmission properties) depending on whether the sensing material 103 is in the presence or liquid (e.g., wet) or in the absence of liquid (e.g., dry), on whether the pH is a certain value (or range of values), or both; the change in light sensitive properties can change upon the change in wavelength of the light. For example, in sensing material 103, one or more light transmission properties of the sensing material 103 change based on exposure to liquid(s) and/or the pH of the exposed liquid(s). In certain embodiments, the sensing material 103 may include a porous medium, such as a mesh or open-cell foam, in which the sensing material may be incorporated; the extent of such incorporation does not substantially change after washing/drying the fabric (e.g., after washing 1, 2, 3, 4, 5, 6, 7, 8, 9, 10, 15, 20, 25, 30, 40, 50, or 100 times). In certain embodiments, the liquid sensing material may include one or more liquid sensing materials (e.g., hydrochromic inks). There are many known liquid sensing materials (e.g., many known hydrochromic inks); any suitable liquid sensing material (e.g., hydrochromic ink) can be used herein, including those known in the art. In other embodiments, the sensing material may include one or more pH sensing materials. There are many known pH sensing materials (e.g., see en.wikipedia.org/wiki/PH_indicator (last accessed Jan. 12, 2025)); any suitable pH sensing material can be used herein, including those known in the art. In yet other embodiments, the sensing material can comprise one or more liquid sensing materials, one or more pH sensing materials, or a combination thereof.

When a light transmission property of the sensing material 103 changes, the characteristics of light passing through the material 103 and collected by light collecting part 102 also change. The light characteristics detected by one or more light detectors 106 correspond with the change resulting from the sensing material 103. Using changes in the data (e.g., light characteristics) from light detectors 106, an electronic circuit 107 is able to make a determination whether or not the sensing material 103 is in the presence of liquid (e.g., wet) or in the absence of liquid (e.g., dry), and/or in some embodiments, if in the presence of liquid (e.g., wet) whether or not the sensing material 103 is a within a certain preselected pH range, or below or above a preselected pH threshold. The at least one electronic circuit 107 is configured to determine a presence or absence of liquid and/or a pH of a present liquid based on a comparison of detected light intensity (e.g., transmission of light) and/or spectra characteristics (e.g., transmission of light at different wavelengths) measured at light detector(s) 106 with an initial light intensity and/or spectra (e.g., light transmissions at one or more wavelengths) characterizing the light emitted from the light emitting part 101. In some embodiments one or more sensors may be included to measure the characteristics of the light emitted by light source 105 prior to any significant changes in the originally produced light. Alternatively, the characteristics of the light from light source 105 may be known from the manufacturer or an initial calibration that does not require repeated measuring of light source 105 over a course of use of the device 100.

In order to communicate the determination of the circuit 107, the device 100 may include at least one output device 108 configured to show different visual appearances when the presence of liquid is determined versus when an absence of liquid is determined, and/or when the determined pH exceeds a predetermined threshold versus when the determined pH does not exceed a predetermined threshold. Non-limiting exemplary output devices may include one or more light sources such as LEDs. The on/off status or a color of one or more LEDs may be used to convey the determination of the electronic circuit 107.

FIG. 1B shows a device 100′ which functions and has utility substantially aligned with the above description of device 100. A distinction, however, is the configuration for the light path. In the case of device 100 (FIG. 1A), the light emitting part 101 and light collecting part 102 may be, for example, two separate optical waveguides arranged in parallel with one another for at least some of their lengths. Furthermore, the parts 101 and 102 are configured to emit or collect light through sides of the waveguides. By contrast, in device 100′, light emitting part 101′ and light receiving part 102′ are arranged end-to-end with one another with the gap containing material 103 positioned between the opposed ends. For example, the light emitting part 101′ and light receiving part 102′ may be configured as two segments of optical waveguide (e.g., optical fiber) which are substantially coaxial with one another in a region of the gap with material 103.

Devices 100 and 100′ may further include one or more nonwovens, wovens, and/or textiles 110 in which the light conducting parts 101/101′ and 102/102′ are embedded or to which they are attached. Accordingly, the devices 100/100′ may include or be configured as bedding and/or an absorbent pad.

A device 100/100′ may not include one or more, or all of, light source 105, light detector(s) 106, electronic circuit(s) 107, and output device(s) 108. These elements may be configured together as one or more modules which can be attached and detached repeatedly with light conducting parts 101/101′ and 102/102′, for example, by connection with one or more terminals/couplers 111 permanently integrated/attached with the material 110. This arrangement advantageously allows the material 110 to be washed for re-use without any risk of exposing electrical elements to water. The washable device 100/100′ has no electrical components when configured to include elements 110, 103, 101/101′, 102/102′, and 111, but to exclude light sources, sensors, circuits, and electrical output devices.

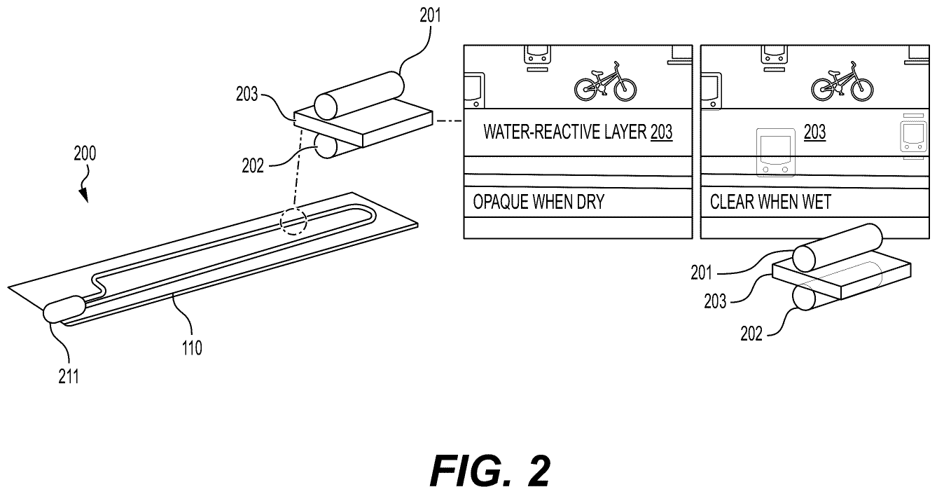

FIG. 2 depicts an exemplary device 200 generally consistent with device 100 of FIG. 1A. FIG. 2 provides further specifics none, some, or all of which may be employed in various embodiments in practice.

FIG. 2 shows a source fiber 201 (i.e., fiber coming from the light source; this is an example of a light emitting part 101) and detector fiber 202 (i.e., fiber going to the detector; this is an example of a light collecting part 102) separated by a layer 203 that switches from opaque when dry to transparent when wet. This transparent textile layer 203 allows light to pass from the source fiber 201 to detector fiber 202, increasing the signal at the detector. The optical fibers 201 and 202 in FIG. 2 may be, for example, stretchable optical fibers, a non-stretchable polymer fiber, a glass optical fiber, and/or another material. In some embodiments, for example for comfort in furniture, bedding, and wearable applications, it can sometimes be desirable for some applications that the fiber be configured to be soft, thin, flexible, or a combination thereof. In certain embodiments, the side of each fiber facing the water-reactive layer 203 may be unclad for stronger inter-fiber light coupling.

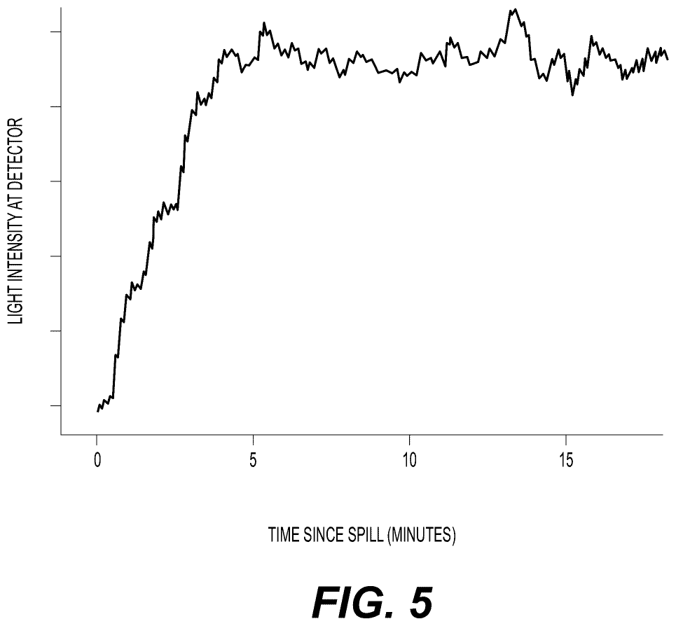

As non-limiting but exemplary aspects of a device like device 200 in FIG. 2, the fibers may be stretchable urethane fibers with diameter of, for example, 1 mm diameter. The water-reactive mesh may be a tulle fabric with, e.g., approximately 400 micron diameter gap filled with a ink such as Matsui Hydrochromic White ink (River City Supply) using a film applicator (available commercially from, e.g., Elcometer USA). The light source and detector connectable at terminal 211 may be a VL53L0x LiDAR chip (ST Microelectronics). The fibers 201 and 202 may be stitched into, e.g., a 3 mm thick foam sheet using a sewing machine with couching foot. The groove created by stitching the light detector fiber 201 into the foam may be used to help align the “light source”/light emitting fiber on the top side of the water-reactive mesh 203. The Example section below and FIG. 5 present performance information for a device reflecting these exemplary characteristics.

The size of the layouts in FIG. 2 may vary based on the intended use of the product. Where lengths are greater than 50 cm, more transmissive fiber materials may be selected. For instance, commercially available PMMA is more transmissive than polyurethane, though either material may be used when fiber lengths are 50 cm or less. Commercial products may include but are not limited to bedding and wearable applications. Products may be configured for specific spill-sensing applications.

For clarity of illustration, FIGS. 1A and 1B each include a single path consisting of a light emitting part 101/101′, a material 103, and a light collecting part 102/102′. In alternative embodiments, however, a single sheet of bedding, pad, or other textile, woven, or nonwoven and the like may contain multiple such paths each positioned differently within the item 110. In such case the material 103 of each respective path is also positioned at a different location within the item 110. In this way, the resolution of wetness/pH detection for device 100/100′ may be made smaller/finer. That is to say, the presence or absence of liquid, and/or pH, may be assessed for different regions of a single fabric/cloth/textile 110. Even in the case of a single light path, the location of the material 103 may be placed at different positions of fabric 110 from those which are illustrated in FIGS. 1A or 1B.

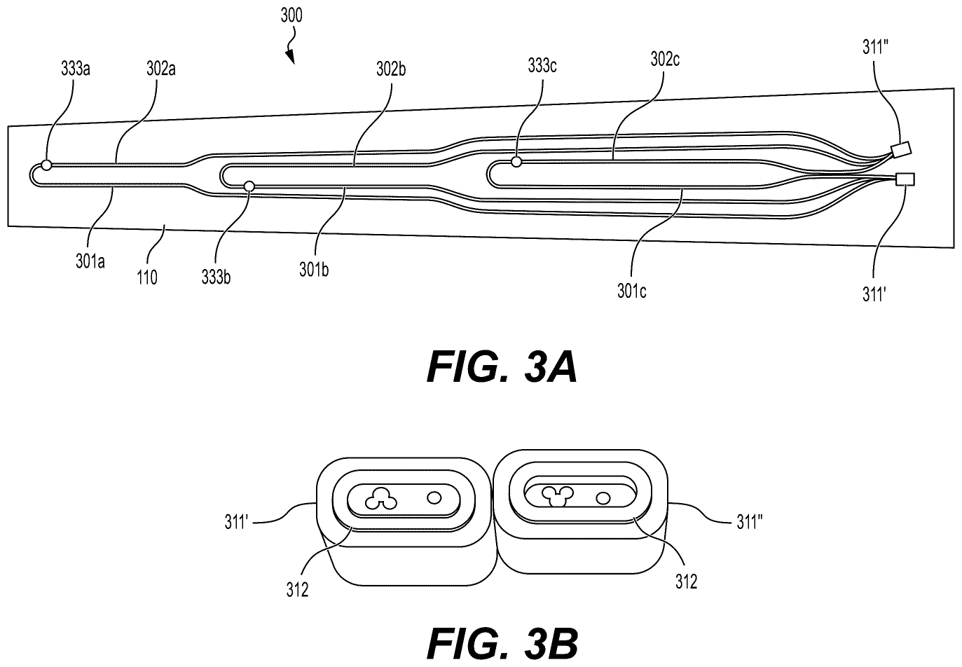

FIG. 3A is a further exemplary embodiment entailing a multi-zone sensor device/system for wider area detections. The device 300 comprises three separate light paths, all of which are affixed to the textile 110. Each of the three light paths includes a pair of waveguides joined by a housing (in this case, shaped as a button to be small and unobtrusive) that houses a liquid-sensitive material, consistent with the description above of FIG. 1B. A light admittance terminal/coupler 311′ is configured to connect to a light source, such as one or more LEDs, or an external waveguide delivering light from such a source to the device 300. From terminal 311′ light enters the light emitting parts 301a, 301b, and 301c. Each of these conduct light to their opposite ends situated in the housings 333a, 333b, and 333c. Each button housing is positioned at a different part of the textile 110, with a result that the device 300 has three independent detection zones. The light reaching each respective button housing may or may not be altered after it is emitted from the waveguide 301a, 301b, or 301c into the liquid-sensitive material within the respective button housing. Light from the opposite side of the liquid-sensitive material is collected by the respective light collecting part 302a, 302b, or 302c, which guides the collected light to the sensor/detector terminal 311″. The terminal 311″ is connectable to sensors which quantify attributes of the light, such as its intensity or spectral characteristics (e.g., light transmissions at one or more wavelengths).

FIG. 3B shows two exemplary couplers usable as terminals 311′ and 311″ to interface the multi-zone fiber device 300 with the light source such as a discrete LED (e.g., white or tri-color) or one or more sensors. The terminals/couplers may include reversible attachment elements such as magnets or magnetic metal 312.

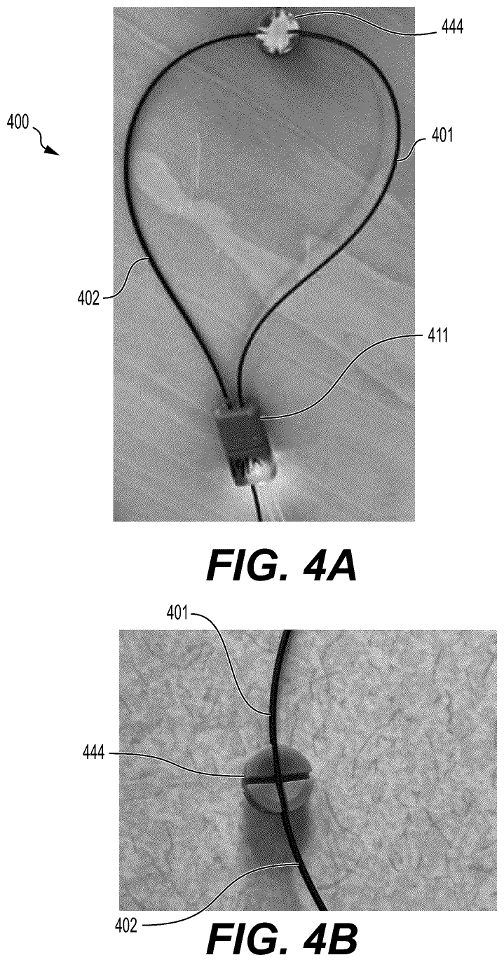

FIG. 4A shows a single light path device 400 which is not attached or incorporated into a textile or fabric, though such option exists. In this embodiment, a single terminal/coupler 411 is provided which is configured to connect the light path formed by waveguides 401 and 402 to both a source of light and to sensors for analyzing the light exiting the light path. Like the arrangement in FIG. 3A, the first waveguide 401 and second waveguide 402 meet end-to-end in a button 444 with a small gap between the adjacent waveguides ends in which a liquid-sensitive material is positioned and through which light from first waveguide 401 must pass to reach second waveguide 402.

FIG. 4B shows a close up of the housing 444 which helps join and keep aligned waveguides 401 and 402. At a minimum, a housing 444 or 333 is a physical body, such as a housing or button, which keeps the two waveguide segments aligned with one another with the liquid sensitive material situated between the ends of the optical waveguide segments.

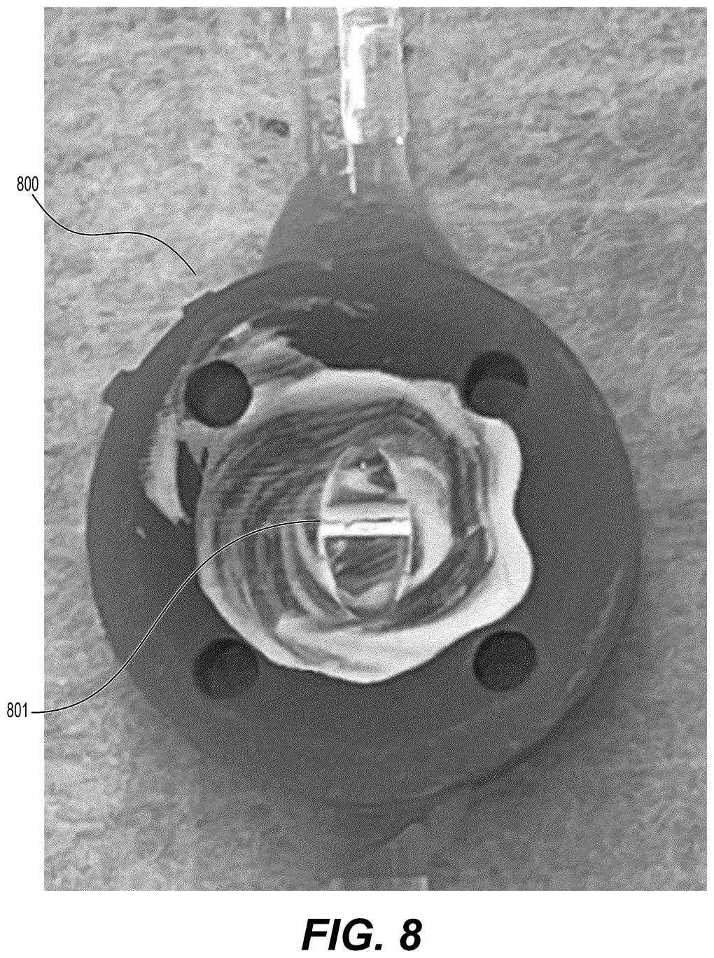

FIG. 8 shows a close-up of another example of a housing (800) in which two fiber ends are in proximity in a dry configuration (801).

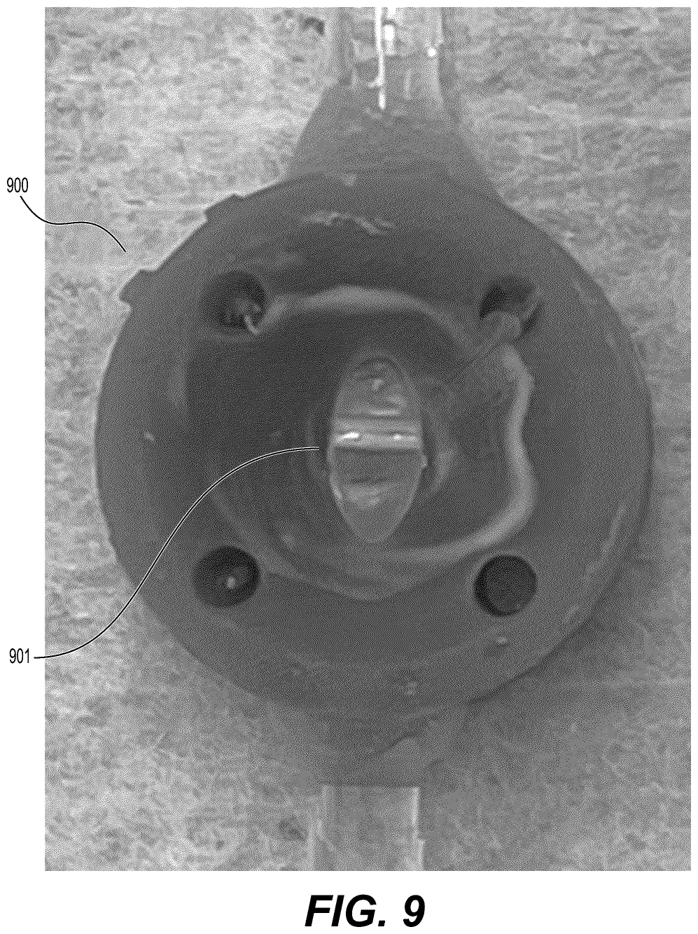

FIG. 9 shows a close-up of another example of a housing (900) in which two fiber ends are in proximity in a wet configuration (901). This is the same housing as that in FIG. 8, except in a wet configuration.

Many alternative materials may be employed for liquid-sensitive materials used in any of the above-described embodiments. The one or more light transmission properties which may be subject to change in liquid-sensitive materials of various embodiments may comprise one or more of opacity, transparency, travel time, and spectral characteristics (e.g., color(s), light transmissions at one or more wavelengths or the product of light transmissions from one or more wavelengths). Some changes may be with respect to particular wavelengths of light. For instance, in some embodiments, a desirable quality is a material that becomes more transparent when in the presence of liquid (e.g., wet) and more opaque when in the absence of liquid (e.g., dry). Such materials may include but are not limited to hydrophilic porous materials and open-cell foams, embedded with, e.g., one or more hydrochromic inks. The liquid-sensitive material which changes light transmission properties depending on exposure to water or other liquids may be implemented as a textile or other water-sensitive film layer as shown in FIG. 2. Alternatively or additionally, the material may be implemented as a coating on one or more optical waveguides or fibers. The gap described above in which the liquid-sensitive material is positioned may correspond with the thickness of the coating. Various optical coatings lend liquid (e.g., via humidity and/or moisture) sensing properties to optical fibers; such coatings may be or include one or more of tungsten disulfide, reduced graphene oxide, and zinc oxide nanoparticles to modify fibers' light transmission properties variably dependent on even small amounts of liquid exposure (e.g., moisture and/or increases in humidity).

Some embodiments may be configured to at least detect a pH (e.g., a pH range) of a liquid to which the device is exposed. The pH detection may be achieved through changes in optical transmission through a coating on an optical fiber gap that is exposed to fluid, consistent with the description above. The liquid-sensitive material can sometimes be chosen so that optical changes (e.g., light transmission properties) occur at specific wavelengths. A pH determination may be made based on differences in detected intensities of particular wavelengths or frequencies (or wavebands or frequency bands) depending on whether or not the liquid-sensitive material is exposed to a liquid of one pH versus a liquid of some different pH.

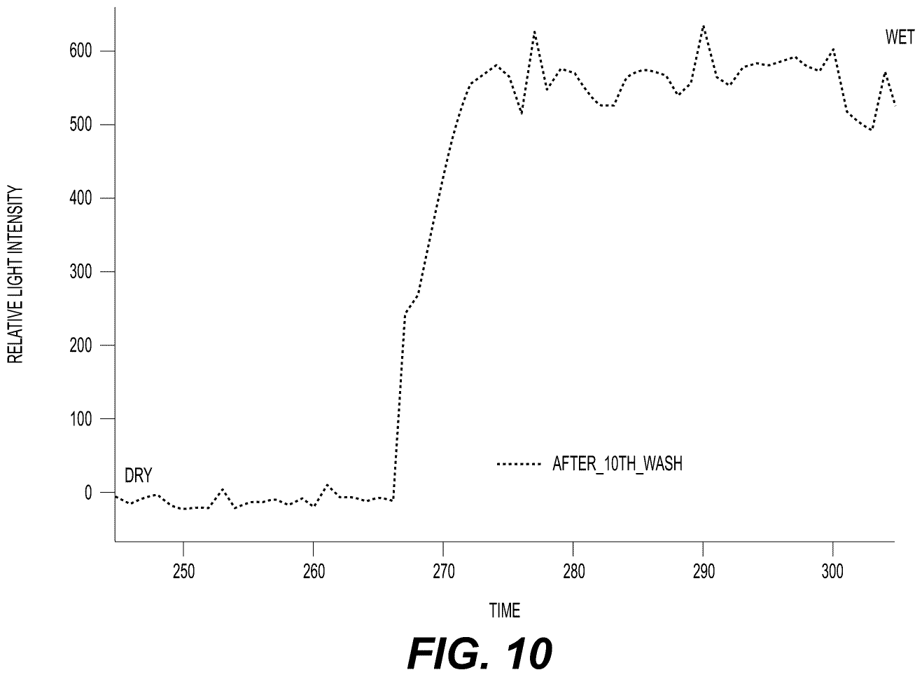

Methods to monitor multiple wavelengths include spectroscopic analysis of transmitted light from a white light source, and sequential analysis of the transmitted intensity from different wavelength light sources (typically discrete light emitting diodes). To eliminate the need for a spectrometer, the light source of some embodiments may be a multiwavelength source, such as a tri-color LED, or a combination of three LEDs, one red, one blue, and one green. Whether one or more LEDs is used, each of multiple different wavelengths (e.g., colors)—such as but not necessarily limited to the primary colors red, green, and blue—may be flashed in sequence (e.g., at 1 or 2 second intervals), allowing for discrete detection of light transmission properties (e.g., light transmission intensity) for each respective wavelength (e.g., color). The liquid-sensitive material is selected such that the spectra (e.g., in the case of emitting and assessing a continuous range of colors simultaneously) or the combination of light intensities (e.g., light transmissions) for multiple discrete colors (i.e., light wavelengths), changes in dependence on pH of the liquid to which the liquid-sensitive material is exposed and through which initial light must pass before being detected and assessed. In some embodiments, the one or more light transmission properties for a multiwavelength source can be the product of the light transmissions of one or more wavelengths; for example, if a blue and green LED are used as light sources, a light transmission property could be the product of the light transmission intensity resulting from the blue LED multiplied by the light transmission intensity resulting from the green LED (see, for example FIG. 10). These features can sometimes provide an economic and compact pH detection method.

Any suitable liquid-sensitive material can sometimes be used also as a pH indicator. One exemplary liquid-sensitive material usable as a pH indicator may be or include bromothymol blue, which responds to increasing pH by shifting its absorbance peak from short to long wavelengths. In some embodiments, the liquid-sensitive material may be a porous hydrochromic ink mixed with, for example, bromothymol blue at 0.1% by weight. Used as a colorimetric indicator for pH above 8, bromothymol blue changes from yellow to blue as the pH increases from below 8 to above 8; accordingly, the circuit of the device can determine whether the pH of a liquid to which the sensor is exposed has a pH below 8 or above 8 based on what color is detected by the light detector and provide such information via the output device. The wavelengths monitored and expected change in transmission are determined by the pH indicator used. In some embodiments, the percent increase or decrease in transmission (i.e., light transmission intensity) at a given wavelength that occurs in detecting the change in pH can be any suitable percent increase or decrease (e.g., as permitted by signal-to-noise), including, for example an increase or decrease of transmission of 1%, 5%, 10%, 20%, 30%, 40%, 50%, 75%, 100%, 125%, 150%, 175%, 200%, 250%, 300%, 350%, 400%, 500%, 600%, 700%, 800%, 900%, 1000%, 1500%, or 2000%. Also, as discussed above, in some embodiments, the one or more light transmission properties for a multiwavelength source can be the product of the light transmissions of one or more wavelengths; for example, if a blue and green LED are used as light sources, a light transmission property could be the product of the light transmission intensity resulting from the blue LED multiplied by the light transmission intensity resulting from the green LED (see, for example FIG. 10). The response time (i.e., the amount of time for the light transmission to reflect the pH of the liquid) can be dependent on many factors including but not limited to the composition of the sensing material, the optical waveguides used, the detectors, or a combination thereof. Some examples of response times include, 1 second, 5 seconds, 10 seconds, 15 seconds, 20 seconds, 30 seconds, 45 seconds, 1 minute, 2 minutes, 3 minutes, 4 minutes, 5 minutes, 6 minutes, 7 minutes, 8 minutes, 9 minutes, 10 minutes, 15 minutes, 30 minutes, 45 minutes, 60 minutes, 90 minutes, 120 minutes, or 240 minutes.

In certain embodiments, the device is configured to distinguish the presence or absence of liquid (e.g., wet/dry conditions) as well as pH, the device may be configured to measure and analyze transmission signal ratio for the presence or absence of liquid (e.g., wet/dry) determination and ratios at different wavelengths for the pH determination. The transmission signal ratio of the liquid-absence/liquid-presence (e.g., dry/wet ratio) may vary from 0.1 to 0.6, for example, depending on the wavelength being monitored. Alternatively, the percent increase in transmission in detecting a sensing material in the absence of liquid (e.g., dry) to detecting a sensing material in the presence of liquid (e.g., wet) can be any suitable percent increase (e.g., as permitted by signal-to-noise), including, for example an increase of transmission of 1%, 5%, 10%, 20%, 30%, 40%, 50%, 75%, 100%, 125%, 150%, 175%, 200%, 250%, 300%, 350%, 400%, 500%, 600%, 700%, 800%, 900%, 1000%, 1500%, or 2000%. The response time (i.e., the amount of time from the transmission reflecting a dry sensing material to the transmission reflecting a wet sensing material) can be dependent on many factors including but not limited to the composition of the sensing material, the optical waveguides used, the detectors, or a combination thereof. Some examples of response times include, 1 second, 5 seconds, 10 seconds, 15 seconds, 20 seconds, 30 seconds, 45 seconds, 1 minute, 2 minutes, 3 minutes, 4 minutes, 5 minutes, 6 minutes, 7 minutes, 8 minutes, 9 minutes, 10 minutes, 15 minutes, 30 minutes, 45 minutes, 60 minutes, 90 minutes, 120 minutes, or 240 minutes.

As an exemplary method for producing a device such as those illustrated in the figures and described above and in the Examples below, may be or include some or all of the following. An optical waveguide (e.g., a plastic optical fiber) is modified with one or more cuts along its length, and the faces of the cut fiber are held in substantially co-axial alignment using a button-shaped housing or housing of other shape which is unobtrusive in size and shape if integrated into textiles, fabrics, wovens, nonwovens, etc. An optical waveguide (e.g., a plastic optical fiber) can be further secured to the housing (e.g., button shaped housing) using any suitable method (e.g., one or more of liquid curing, epoxy, silicone, or injection molding), which can sometimes result in an increase in the number of times the housing/sensor can washed and reused. A porous liquid sensing compound (e.g., hydrochromic ink) mixed with a pH sensitive compound (e.g., bromothymol blue at 0.1% by weight) if pH sensitivity is desired, is painted over a gap in the housing which permits external liquid to reach the ink. For instance, such a gap is visible in the housing 444 of FIG. 4B, in housing 800 of FIG. 8 (see where two fiber ends are in proximity in a dry configuration—801) and in housing 900 (see where two fiber ends are in proximity in a wet configuration—901); the two free ends of the optical waveguide can be connected to a single terminal (e.g., terminal 411 in FIG. 4A) or each to its own respective terminal (e.g., terminals 311′ and 311″ of FIGS. 3A/3B). Finally, an optical waveguide, housing with hydrochromic ink, and terminal(s) may be attached or integrated into a fabric, textile, woven, nonwoven, etc. for which the functionality of detecting the presence of liquids and/or pH is desired, e.g., bedding, incontinence pads, etc. Coupler(s)/terminal(s) of the device can sometimes be attached to an one or more of external light source, light detector, decision-making circuitry, and output devices, as discussed in connection with FIGS. 1A and 1B above, to monitor the optical transmission through the fiber over a range of wavelengths, make determinations of the active condition of the sensor (and correspondingly any change in condition), and provide this information to an external device or user.

The presently-disclosed subject matter is further illustrated by the following specific but non-limiting examples. The following examples may include compilations of data that are representative of data gathered at various times during the course of development and experimentation related to the present invention.

### EXAMPLES

### Example 1. Liquid Detection Using Hydrochromic Ink

An embodiment was prepared consistent with the general disclosure of FIG. 2 and its accompanying description above. The fibers were StretchMagic™ 1 mm diameter urethane fibers (Pepperell, Inc). The water-reactive layer was a tulle fabric mesh with approximately 400 micron diameter openings filled with Matsui Hydrochromic White ink (River City Supply) using a film applicator (Elcometer USA). The source and detector were a VL53L0x LiDAR chip (ST

Microelectronics). The fibers were stitched into a 3 mm thick foam sheet using a sewing machine with couching foot. The groove created by stitching the light detector fiber into the foam helped align the “light source” fiber on the top side of the water-reactive mesh. In response to a 10 ml water spill, the detected light intensity increased by 50% (from 50 mega counts per second to 75 mcps) over a 5-minute period, as shown by the plot of FIG. 5.

### Example 2. pH Detection

A device was prepared by modifying a plastic optical fiber (such as Duraband or TPU) with a cut and fixing the resulting faces from the cut close together and in co-axial alignment using a button-shaped, 3D printed housing (e.g., see FIGS. 4A and 4B). An extra slot was added perpendicular to the fiber slot; the fiber slot is open so the optical fiber can be placed, glued and then cut with a razor blade through that extra slot, potentially making it easier to install with automation. A porous hydrochromic ink mixed with bromothymol blue at 0.1% by weight was painted over and within the gap and allowed to dry. The dried coating was then exposed to fluids while the optical transmission through the fiber over a range of wavelengths was monitored. Long used as a colorimetric indicator for pH above 8, bromothymol blue changes from yellow to blue as the pH increases.

The transmission signal ratio of dry/wet varies from 0.1 to 0.6 depending on the different wavelengths before and after washes. The ratios at different wavelengths indicate the filled agent in the sensor due to the acidity.

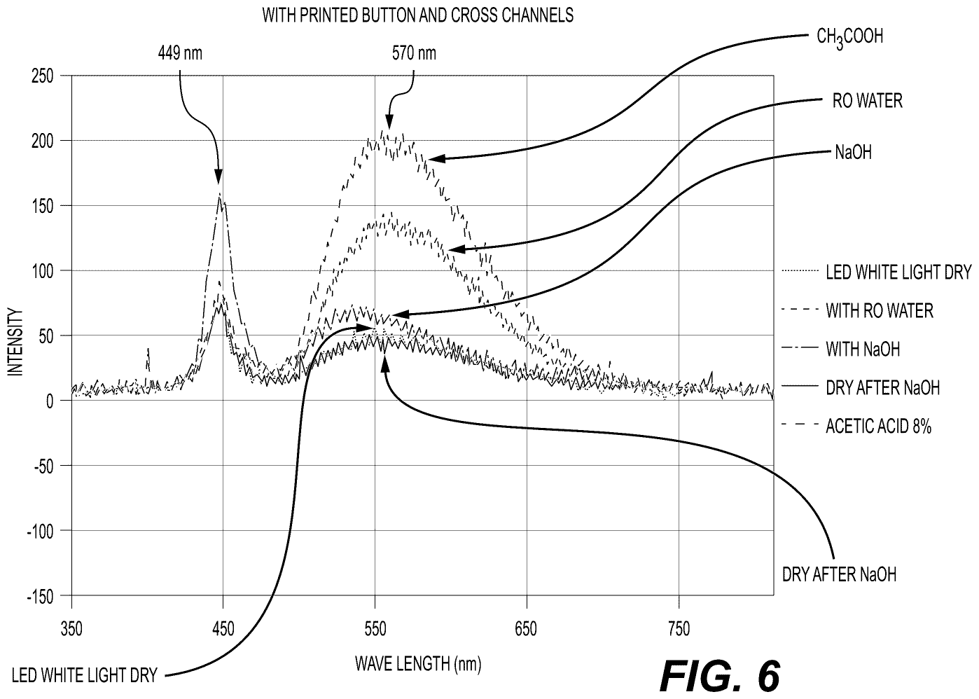

FIG. 6 shows resulting transmission spectroscopy with a white light emitting diode (LED) light source. The data indicates the acidity of the fluid in the sensor. Neutral pH reverse osmosis (RO) water increases transmission above the dry value by wetting the hydrochromic ink, showing how the button functions as a liquid sensor regardless of pH. Acetic acid (CH₃COOH) at 8% (v/v) has a pH of about 2.3 and increases the transmission at 600 nm of the bromothymol blue pH indicator. Sodium hydroxide (NaOH) at 1M has a pH of about 13 and decreases the transmission at 600 nm of the bromothymol blue indicator (nearly to the dry level), but also increases its transmission at 450 nm. Notably, the two dry curves (LED white light dry and Dry after NaOH) are virtually identical. These results demonstrate the sensor was able to distinguish dry, wet, acidic, and basic (defined in this instance as pH>8) conditions.

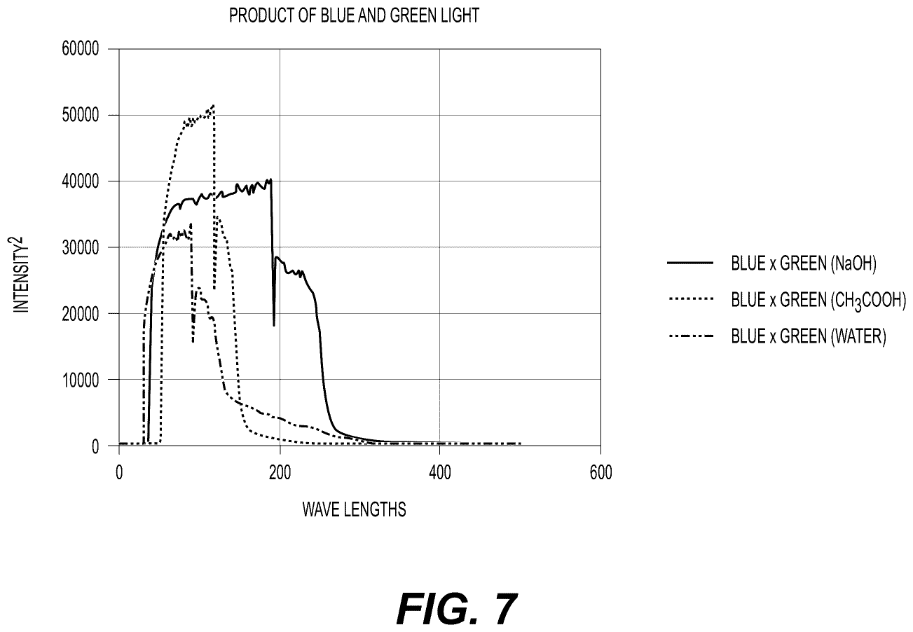

To eliminate the need for a spectrometer, a discrete detection method was implemented using a tri-color LED as the light source, with respective primary colors emitted in sequence to one another. The detected signals from the sensor detecting the light the tri-color LED after it passed through the hydrochromic ink was analyzed. FIG. 7 shows the results from exposing the device to water, acidic (CH₃COOH), and basic (NaOH) fluids. The y-axis is a product of the blue LED intensity multiplied by the green LED intensity.

### Example 3. Sensor Button with Alternative Geometry

In this example, a device was prepared by modifying a plastic optical fiber (such as Duraband or TPU) with a cut before the fibers were inserted from the sides of the button, fixing the resulting faces from the cut close together and in substantial co-axial alignment using a button-shaped, 3D-printed housing (˜10 mm diameter). The button holds the two fiber ends in proximity to create a gap. Hydrochromic ink was painted over and within the gap. FIG. 8 shows the device when dry and FIG. 9 shows the device when wet. Of course, this device configuration can be used to detect either liquid or pH.

The sensor button in this example has a thinner gap compared to the device in Examples 1 and 2; this thinner gap provides a faster response time when exposed to liquid. For example, in FIG. 10, the response time when the sensor button in this example is exposed to water is within 20 seconds, as compared to about 3-5 minutes for the sensor button of Example 1.

“Circuits” and “circuitry” as used in this disclosure may refer to purely analog electronics, digital electronics, or a combination thereof. Depending on the embodiment, circuits and circuitry may include or exclude one or more processors like microprocessors or central processing units (CPUs) or other computing devices. Circuits and circuitry may include one or more computer readable storage media having computer readable program instructions thereon for causing a processor executing such instructions to carry out aspects of embodiments described herein.

A computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, or either source code or object code written in any combination of one or more programming languages, including an object oriented programming language such as Java, Smalltalk, C++ or the like, and conventional procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

The headings used in the disclosure are not meant to suggest that all disclosure relating to the heading is found within the section that starts with that heading. Disclosure for any subject may be found throughout the specification.

It is noted that terms like “preferably,” “commonly,” and “typically” are not used herein to limit the scope of the claimed invention or to imply that certain features are critical, essential, or even important to the structure or function of the claimed invention. Rather, these terms are merely intended to highlight alternative or additional features that may or may not be utilized in a particular embodiment of the present invention.

As used in the disclosure, “a” or “an” means one or more than one, unless otherwise specified. As used in the claims, when used in conjunction with the word “comprising” the words “a” or “an” means one or more than one, unless otherwise specified. As used in the disclosure or claims, “another” means at least a second or more, unless otherwise specified. As used in the disclosure, the phrases “such as”, “for example”, and “e.g.” mean “for example, but not limited to” in that the list following the term (“such as”, “for example”, or “e.g.”) provides some examples but the list is not necessarily a fully inclusive list. The word “comprising” means that the items following the word “comprising” may include additional unrecited elements or steps; that is, “comprising” does not exclude additional unrecited steps or elements.

As will be apparent to those of skill in the art upon reading this disclosure, each of the individual embodiments described and illustrated herein has discrete components and features which may be separated from or combined with the features of any of the other several embodiments without departing from the scope or spirit of the present invention. Any recited method can be carried out in the order of events recited or in any other order which is logically possible.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are described.

Detailed descriptions of one or more embodiments are provided herein. It is to be understood, however, that the present invention may be embodied in various forms. Therefore, specific details disclosed herein (even if designated as preferred or advantageous) are not to be interpreted as limiting, but rather are to be used as an illustrative basis for the claims and as a representative basis for teaching one skilled in the art to employ the present invention in any appropriate manner. Indeed, various modifications of the invention in addition to those described herein will become apparent to those skilled in the art from the foregoing description and the accompanying figures. Such modifications are intended to fall within the scope of the appended claims. While exemplary embodiments of the present invention have been disclosed herein, one skilled in the art will recognize that various changes and modifications may be made without departing from the scope of the invention as defined by the following claims.

What is claimed is:

1. A washable and re-usable sensing device, comprising
a light emitting part;
a light collecting part;
a gap separating a portion of or all of the light emitting part and a portion of or all of the light collecting part, from one another;
a liquid-sensitive material in the gap, wherein one or more light transmission properties of the liquid-sensitive material change based on exposure to a liquid and/or pH of a present liquid;
wherein the light collecting part is configured to collect light emitted from the light emitting part which has passed through the liquid-sensitive material and transmit the collected light to at least one terminal configured to connect the light collecting part to one or more light detectors.

2. The washable and re-usable sensing device of claim 1, further comprising the one or more light detectors, wherein the one or more light detectors are configured to detect one or more of light transmissions at one or more wavelengths.

3. The washable and re-usable sensing device of claim 2, further comprising at least one electronic circuit configured to determine a presence or absence of liquid and/or a pH of a present liquid based on a comparison of detected light transmissions at one or more wavelengths with an initial light transmission at one or more wavelengths characterizing the light emitted from the light emitting part.

4. The washable and re-usable sensing device of claim 3, further comprising at least one output device configured to show different visual appearances when the presence of liquid is determined versus when an absence of liquid is determined, and/or when the determined pH exceeds a predetermined threshold versus when the determined pH does not exceed a predetermined threshold.

5. The washable and re-usable sensing device of claim 1, wherein
(1) the light emitting part and light collecting part are optical fibers, and
(2) (a) the two optical fibers are arranged so that a portion of or all of one or both optical fibers are in parallel in a region of the gap and/or (b) the two optical fibers are substantially coaxial with one another in a region of the gap.

6. The washable and re-usable sensing device of claim 1, wherein the liquid-sensitive material comprises a washable hydrochromic ink.

7. The washable and re-usable sensing device of claim 1, wherein the liquid-sensitive material comprises one or more of a mesh, at least one hydrophilic porous material, or at least one open-cell foam.

8. The washable and re-usable sensing device of claim 1, further comprising one or more textiles and/or nonwovens in which the light emitting part and light collecting part are embedded.

9. The washable and re-usable sensing device of claim 1, wherein the one or more light transmission properties comprise one or more of opacity, transparency, light transmission(s) at one or more wavelengths, the product of light transmissions from more than one wavelength, travel time, or color.

10. A method of sensing a presence of liquid and/or a pH or pH range of a present liquid, comprising
emitting light from a light emitting part toward a light collecting part, wherein a gap separates a portion of or all of the light emitting part and a portion of or all of light collecting part from one another, wherein a liquid-sensitive material is positioned in the gap, wherein one or more light transmission properties of the liquid-sensitive material change based on exposure to the liquid and/or the pH of the liquid;
detecting one or more of light transmissions at one or more wavelengths of light collected by the light collecting part;
determining a presence or absence of liquid and/or a pH or a pH range of a present liquid based on a comparison of detected light transmissions at one or more wavelengths with an initial light transmission at one or more wavelengths characterizing the light emitted from the light emitting part; and
showing different visual appearances at one or more output devices
(a) when the presence of liquid is determined, as compared to when an absence of liquid is determined, and/or
(b) when (1) the determined pH is in a predetermined pH range and/or (2) the determined pH exceeds a predetermined threshold, as compared to when the determined pH does not exceed a predetermined threshold and/or (3) the determined pH is below a predetermined threshold, as compared to when the determined pH is not below a predetermined threshold.

11. A washable and re-usable liquid sensing device, comprising
at least one first optical waveguide configured to transmit light from at least one light source and emit some of the transmitted light through a side or end of the at least one first optical waveguide;
at least one second optical waveguide;
a liquid-sensitive material positioned between the at least one first optical waveguide and the at least one second optical waveguide such that light emitted from at least a portion of the at least one first optical waveguide must pass through the liquid-sensitive material to reach at least a portion of the at least one second optical waveguide, wherein one or more light transmission properties of the liquid-sensitive material change based on the exposure of the liquid-sensitive material,
wherein the at least one second optical waveguide is configured to collect incident light emitted from the at least one first optical waveguide which has passed through the liquid-sensitive material and transmit the collected light to at least one light detector terminal.

12. The washable and re-usable liquid sensing device of claim 11, further comprising a terminal configured for connection to a light source for admitting light into the at least one first optical waveguide and to a detector for receiving light exiting an end of the at least one second optical waveguide.

13. The washable and re-usable liquid sensing device of claim 11, further comprising one or more textiles and/or nonwovens in which the at least one first optical waveguide and the at least one second optical waveguide are embedded.

14. The washable and re-usable liquid sensing device of claim 13, configured as a bedding and/or a pad.

15. The washable and re-usable liquid sensing device of claim 11, wherein the liquid-sensitive material is configured as at least one coating of a hydrochromic ink on one or more of the at least one first optical waveguide and the at least one second optical waveguide.

16. The washable and re-usable liquid sensing device of claim 11, wherein the liquid-sensitive material is configured as a film layer.

17. The washable and re-usable liquid sensing device of claim 11, wherein the at least one first optical waveguide and/or the at least one second optical waveguide is optical fiber comprising PMMA or urethane.

18. The washable and re-usable liquid sensing device of claim 11, wherein the liquid-sensitive material comprises one or more of a mesh, at least one hydrophilic porous material, or at least one open-cell foam containing a hydrochromic ink.

19. The washable and re-usable liquid sensing device of claim 11, wherein the liquid-sensitive material comprises one or more of tungsten disulfide, reduced graphene oxide, or zinc oxide nanoparticles.

20. The washable and re-usable liquid sensing device of claim 11, wherein the one or more light transmission properties comprise one or more of opacity, transparency, light transmission(s) at one or more wavelengths, the product of light transmissions from more than one wavelength, travel time, or color.
